SEQUENCE LISTING (2 sequences)

SEQ ID NO: 1 - protein, 9 aa, Inovirus M13
  source 1..9: note = Phage M13
QRKLAAKLT

SEQ ID NO: 2 - protein, 14 aa, Inovirus M13
  source 1..14: note = Phage M13
KLAKLAKKLAKLAK

(19) United States
(12) Patent Application Publication
(10) Pub. No.: US 2024/0353406 A1
(43) Pub. Date: Oct. 24, 2024

(54) INNOVATIVE BIOTECHNOLOGICAL SYSTEMS FOR THE DETECTION OF CELLULAR OR MOLECULAR ANALYTES

(71) Applicants: UNIVERSITA' DEGLI STUDI DI MESSINA, Messina (ME) (IT); ALMA MATER STUDIORUM - UNIVERSITA' DI BOLOGNA, Bologna (IT)

(72) Inventors: Sabrina CONOCI, Messina (ME) (IT); Salvatore GUGLIELMINO, Messina (ME) (IT); Matteo CALVARESI, Messina (ME) (IT); Alberto DANIELLI, Messina (ME) (IT); Luca PRODI, Messina (ME) (IT); Enza FAZIO, Messina (ME) (IT); Annapaola PETROSINO, Messina (ME) (IT); Matteo DI GIOSIA, Messina (ME) (IT)

(21) Appl. No.: 18/693,672

(22) PCT Filed: Sep. 21, 2022
(86) PCT No.: PCT/IB2022/058928

§ 371 Date: Mar. 20, 2024

(30) Foreign Application Priority Data
Sep. 21, 2021 (IT) 102021000024185

(51) Int. Cl. G01N 33/569; G01N 21/64; G01N 33/543

(52) U.S. Cl. CPC G01N 33/56911; G01N 21/6486; G01N 33/54326; G01N 2333/01; G01N 2333/21

(57) ABSTRACT

A method for detecting cellular or molecular analytes is provided. The method uses two types of engineered phages: a “capture” phage (bait phage) and a “signal” phage (reporter phage). Through this method it is possible to selectively bind a target analyte and thus recognize said target analyte.

### TECHNICAL FIELD

The present invention relates to a method for detecting cellular or molecular analytes, which method uses two types of engineered phages: a “capture” phage (bait phage) and a “signal” phage (reporter phage). Through this method it is possible to selectively bind a target analyte and thus recognize said target analyte.

### Background Art

Within the field of laboratory diagnostic systems in general, for example, clinical and molecular diagnostic systems, there is always a strong interest in developing innovative systems.

The main analytical methodologies currently used for detecting cellular systems (microbes and eukaryotic cells) of analytes of different nature (such as viruses, molecular markers, toxins, proteins, nucleic acids, etc.) are mainly based on indirect detection systems, which, though sensitive (for example, PCR, ELISA, other antibody tests, etc.), provide for manipulations by qualified operators, wait times for getting answers from the test, and centralization of the analyses.

Another aspect of interest is the so-called POCT (Point-Of-Care Technologies) technologies, for developing bioengineered systems integrated with innovative biotechnologies. Particularly, such systems allow performing rapid analytical tests in non-laboratory contexts, carried out by non-qualified personnel or even by the patient himself/herself. In particular, the increasing attention to research in the POCT field has been largely driven by two distinct trends in modern society: (i) the general need to reduce high healthcare costs and (ii) the demand for better analytical solutions for early diagnoses and customized therapies. In addition to this, as a result of the burst of the pandemic, the demand for massive screenings, which at the same time should be highly accurate, sensitive and inexpensive, has gained great importance. These aspects are crucial to deal with efficient patient management, timely diagnostics, as well as prompt detection of infectious disease outbreaks, thus making it possible to deal efficiently with pandemic or epidemic events. Therefore, there is still a search going on for analytical systems that are sensitive and selective, and at the same time inexpensive and having direct readout, thus allowing for direct detection of molecular and/or cellular analytes, in particular microorganisms (e.g., bacteria and viruses), in which systems said detection should have high selectivity, specificity and cost-effectiveness.

### SUMMARY OF INVENTION

The problem addressed by the present invention is thus to provide a method for detecting analytes such as, for example, cellular systems (for examples, bacteria, parasites, eukaryotic cells), viruses, molecular markers (for example, toxins, proteins, nucleic acids), preferably of bioclinical interest, which method should have characteristics of high sensitivity and specificity, is direct and can also be used in rapid and cost-effective systems.

This problem is solved by the method of the present invention, as outlined in the appended claims, the definitions contained therein being an integral part of the present disclosure.

The Applicant has now found a method for detecting cellular or molecular analytes that uses two types of engineered phages: a bait phage and a reporter phage. Specifically, said phage comprises at least one fusion peptide exposed on the major capsomer (major coat protein) pVIII and capable of selectively binding the target analyte. Subsequently, the reporter phage exposing at least one recognition peptide on the minor capsomer (minor coat protein) pIII, at the phage tip, and at least one marker for signal transduction on the major capsomer, binds in turn the analyte captured by the bait phage. Such detection system based on the two types of engineered phages can be defined as “molecular sandwich”.

Therefore, in a first aspect, the present invention relates to a method comprising the steps of:
a) contacting a sample containing the analyte with first phages M13 (bait phages) bound to an appropriate support and comprising at least one peptide or polypeptide or fusion protein specific for recognizing the analyte, said peptide or polypeptide or fusion protein being exposed on the protein pVIII, thus achieving formation of first complexes comprising first phages M13 bound to the analyte (“bait phage-analyte” complexes);
b) separating the first phages M13 from the sample part that, in the preceding step a), did not bind to the first phages M13;
c) adding, to the first phages M13, second phages M13 (reporter phages) comprising at least one peptide or polypeptide or protein for specific recognition of the analyte fused on the protein pIII, and at least one marker conjugated to the capsid of said second phages M13, said marker being selected from a fluorophore, a chromophore, an electrochemically active species or an electrochemiluminescence-active species, thus achieving formation of second complexes comprising first phages M13 and second phages M13 bound to the analyte in a sandwiched manner (“bait phage-analyte-reporter phage” complexes);
d) carrying out a washing to remove the second phages M13 that did not bind the analyte,
e) determining the derived signal generated by the markers of the second phages M13 of said second complexes.

Advantageously, the method according to the invention makes it possible to detect a target analyte in a selective manner, with high sensitivity and in a direct manner. Particularly, said method can be used for direct detection of infectious microorganisms (for example, bacteria, parasites and viruses, for example, SARS-CoV-2). Other advantageous examples include direct detection of human eukaryotic cells, as in the case of variations due to pathologies such as oncological pathologies, as well as analytes of different molecular nature (for example, proteins, molecular markers, toxins, nucleic acids, etc.). Advantageously, the method according to the invention provides for the possibility of detections by multiplexing for the integrated analysis of several analytes at the same time. Also, another advantageous aspect is that this type of detection is inexpensive. Finally, implementation of the method of the invention for POCT is also possible.

Further features and advantages of the method of the invention will be apparent from the description of the exemplary embodiments of the invention, provided as indicative examples of the invention itself.

### BRIEF DESCRIPTION OF DRAWINGS

FIG. 1 is a schematic illustration of the mechanism of action of the method according to the invention;

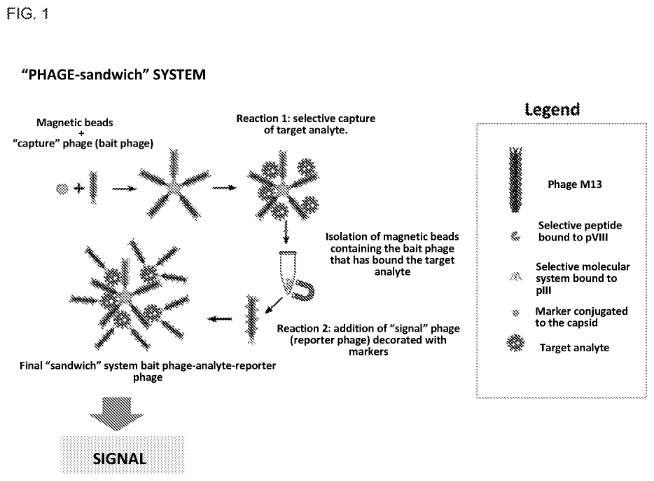

FIG. 2 shows an image of the molecular sandwich “bait phage+analyte+reporter phage”;

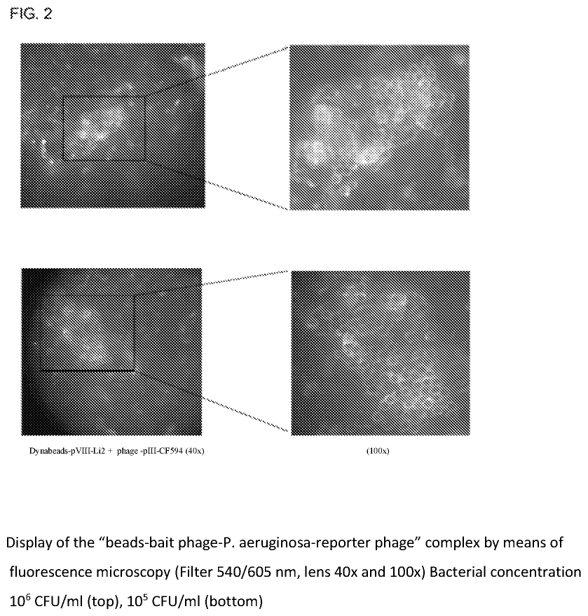

FIG. 3 shows a further image of the molecular sandwich “bait phage+analyte+reporter phage”;

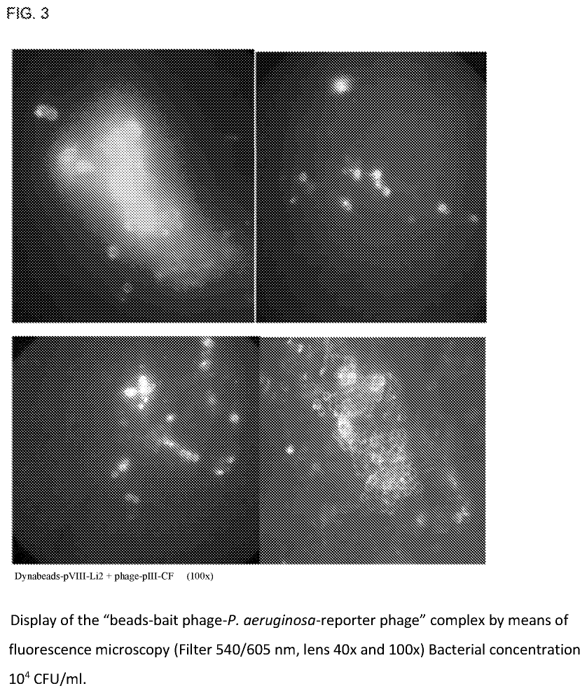

FIG. 4 shows a further image of the molecular sandwich “bait phage+analyte+reporter phage”;

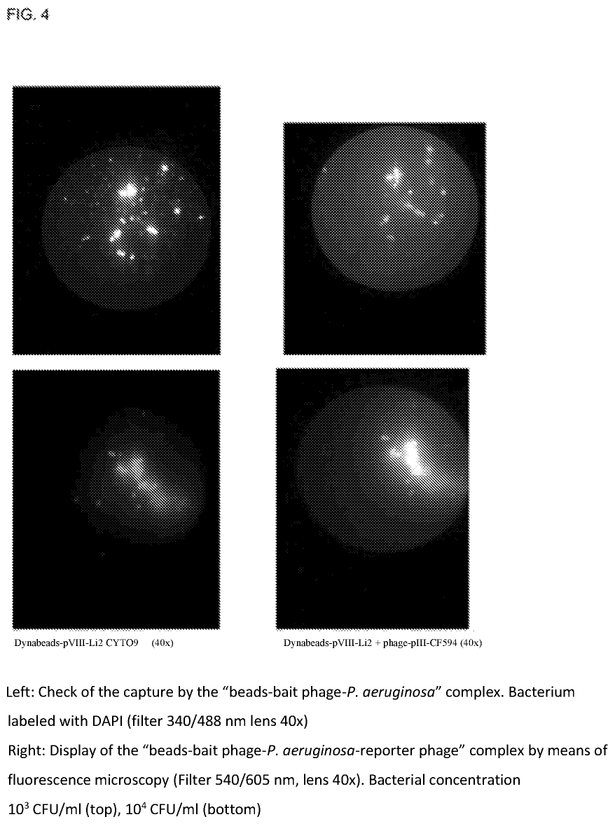

FIG. 5 shows a further image of the molecular sandwich “bait phage+analyte+reporter phage”;

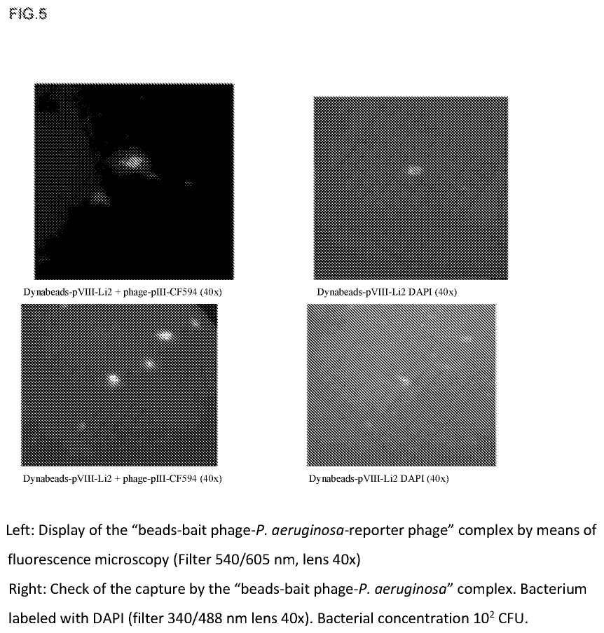

FIG. 6 shows a further image of the molecular sandwich “bait phage+analyte+reporter phage”.

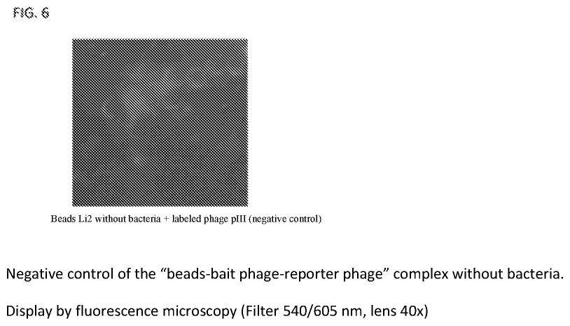

### DESCRIPTION OF EMBODIMENTS

For the purposes of the invention, the definitions of some terms used in the present description and in the appended claims are provided below.

The term “cellular or molecular analyte” means systems such as, for example, cellular systems (such as microbes, parasites, eukaryotic cells), viruses, molecular markers (such as toxins, proteins, nucleic acids, etc.), preferably of bioclinical interest.

The object of the present invention is a method for detecting an analyte in a sample, wherein said analyte is selected from:
a cellular system selected from: microbe, parasite, eukaryotic cell, bacterium, virus; or
a molecular marker selected from: toxin or protein;
   said method comprising the steps of:
a) contacting the sample with first phages M13 (bait phages) bound to an appropriate support and comprising at least one peptide or polypeptide or fusion protein specific for recognizing the analyte, said peptide or polypeptide or fusion protein being exposed on the protein pVIII, thus achieving formation of first complexes comprising first phages M13 bound to the analyte (“bait phage-analyte” complexes);
b) separating the first phages M13 from the sample part that did not bind to the first phages M13;
c) adding, to the first phages M13, second phages M13 (reporter phages) comprising at least one peptide or polypeptide or protein for specific recognition of the analyte fused on the protein pIII, and at least one marker conjugated to the capsid of said second phages M13, said marker being selected from a fluorophore, a chromophore, an electrochemically active species or an electrochemiluminescence-active species, thus achieving formation of second complexes comprising first phages M13 and second phages M13 bound to the analyte in a sandwiched manner (“bait phage-analyte-reporter phage” complexes);
d) carrying out a washing to remove the second phages M13 that did not bind the analyte;
e) determining the derived signal generated by the markers of the second phages M13 of said second complexes.

The above-mentioned steps are carried out in the order set forth above.

Preferably, the first phage M13 (bait phage) is an engineered phase M13 exposing at least one fusion peptide, preferably in fusion with the protein “major coat protein pVIII” of the phage, capable of selectively binding the analyte. Preferably, the at least one fusion peptide is exposed in a number of copies in the order of tens or hundreds, up to thousands, of copies on the capsid of the first phage M13.

The second phage M13 (reporter phage), is a phage M143 engineered with methods known to the person skilled in the art and exposing at least one peptide or polypeptide or fusion protein, preferably in fusion with the “minor coat protein pIII” of the phage, capable of selectively binding the analyte. The protein “minor coat protein pIII” is a protein of the capsid of said second phage M13.

With reference to the marker, it allows determining the derived signal generated by the marker. Such signal determination can take place by optical or electronic detection of the analyte. In particular, the at least one marker can be detected by means of photometry, fluorimetry, photoluminescence, thermoluminescence, chemiluminescence, electrochemiluminescence, voltammetry and other electrochemical techniques. In fact, after the passage at step c), the signal of effected “capture” of the analyte can be detected by means of optical or electronic sensors, depending on the marker used. According to a preferred aspect, the marker conjugated to the capsid of the second phage M13 is a fluorophore, a chromophore, an electrochemically active species or an electrochemiluminescence-active species. According to a preferred aspect of the invention, the marker is a fluorophore, the fluorophore preferably being CF594 (Biotium).

According to a preferred aspect of the invention, the analyte is a bacterium, preferably a Gram-negative bacterium, even more preferably P. aeruginosa. Preferably, the at least one peptide or polypeptide or fusion protein of step a) has the sequence SEQ. ID NO: 1 (QRKLAAKLT). Preferably, the at least one peptide or polypeptide or phase recognition protein of step c) has the sequence SEQ. ID NO: 2 (KLAKLAKKLAKLAK).

According to a preferred aspect of the invention, when the analyte is P. aeruginosa, the at least one peptide or polypeptide or fusion protein of step a) has the sequence SEQ. ID NO: 1 (QRKLAAKLT). Preferably, said at least one fusion peptide is exposed in a number of copies in the order of tens or hundreds, up to thousands, of copies on the capsid of the first phage M13.

According to a preferred aspect of the invention, when the analyte is P. aeruginosa, the at least one peptide or polypeptide or phage recognition protein of step c) has the sequence SEQ. ID NO: 2 (KLAKLAKKLAKLAK).

According to a preferred aspect of the invention, when the analyte is P. aeruginosa, the at least one peptide or polypeptide or fusion protein of step a) has the sequence SEQ. ID NO: 1 (QRKLAAKLT) and the at least one peptide or polypeptide or phage recognition protein of step c) has the sequence SEQ. ID NO: 2 (KLAKLAKKLAKLAK).

According to a preferred aspect, a linker can be used. Advantageously, the linker confers characteristics of mobility to the peptide. Preferably, the linker is GGGS.

In a preferred aspect of the method according to the invention, a support, for example, a magnetic microsphere (MNP), is used, said support being functionalized with a bait phage (thus obtaining an MNP-bait phage conjugate), wherein said phage comprises at least one fusion peptide exposed on the major capsomer (major coat protein) pVIII and capable of selectively binding the target analyte. Also, exemplary but not limiting procedures of functionalization are set forth below. Therefore, in the detection method according to the invention, a support that is functionalized with the first phage M13 can be used. Preferably, the support is a magnetic microsphere (MNP). Herein, the term “magnetic microspheres”, commonly referred to in the art as magnetic microparticles MNP, means particles on which one or more phages can be immobilized. As a person skilled in the art will understand, herein said particles can also be referred to by interchangeably using the term sphere/spheres and corresponding terms (for example, microspheres). Such particles can be inorganic particles, preferably of ferrite or the like, and can be functionalized with one or more phages.

As a result of said functionalization, a biosensor can be obtained. In particular, the microspheres are advantageous because of their tridimensional structure with a ratio between surface area and volume higher than that of a plan surface, thus providing more binding sites for the target. Another advantage is their easiness of handling in fluids, as well as their cost-effectiveness. According to a preferred aspect, the selected phages exposing the active recognition peptides fused to the protein pVIII are covalently bound on the magnetic microspheres. This is of advantage for use in the capture, washing and rapid concentration of cells, viruses or proteins directly in the sample under test.

According to a preferred aspect, if the support used for the first phages M13 consists of magnetic microspheres, the step of separation of the first phages M13 from the sample part not bound to the first phages M13 takes place by means of magnetic capture of the magnetic microspheres.

According to a further preferred aspect, the support for the first phages M13 is an electrode, a polymer or a silicon-based surface. In this case, the step of separation of the first phages M13 from the sample part not bound to the first phages M13 takes place by washing. Preferred applications of the method of the invention are aimed at direct detection of cells of microorganisms with special reference to infectious microorganisms (bacteria and viruses), such as pathogenic Gram-negative bacteria (e.g., species listed in the WHO's “priority list”) or viruses such as SARS-CoV-2. Such method can also be extended to eukaryotic cells, especially in liquid biopsy with the possibility of capturing, concentrating and directly detecting cancer cells. In this case, the selectivity of the method will be related to the specificity of the engineered bait phages and reporter phages towards specific tumor markers exposed on the surface of the cells. Such method can also be extended to molecular analytes, such as toxins, proteins and nucleic acids, especially when these can be considered as biomarkers for certain pathologies. The development of such “phage probes” and, accordingly, the flexibility of the analytical platform presented, is favored by the large adaptability of the phage-display schemes allowing to select, by biopanning, peptide and antibody libraries (scFvs, single-domain antibodies, etc.) following immobilization of the proteins of interest (either native or recombinant) on the aforementioned supports.

The above is to be understood as being exemplary and not limiting. In addition, the person skilled in the art will be able to understand that modifications can be made without departing from the scope of the present invention.

### EXAMPLES

### Reagents Used and Preparation Thereof

TABLE 1
Buffer A: borate buffer 0.1 M, pH 9.5
6.18 g H3BO3 (MW 61.83). Dissolve in 800 mL distilled
water. Adjust the pH to 9.5 by using 5 M NaOH and adjust
the volume to 1 L with distilled water.
Buffer B: 0.1 M Na-phosphate buffer, pH 7.4
2.62 g NaH2PO4. H2O (MW 137.99) and 14.42 g Na2HPO4.
2 H2O (MW 177.99). Adjust the volume to 1 L with distilled
water.
Buffer C: 3 M ammonium sulphate in buffer A or B
39.64 g (NH4)2SO4 dissolved in buffer A or B. Adjust the
pH with NaOH or HCl. Adjust to 100 mL with buffer A or B.
Buffer D
PBS pH 7.4 with 0.5% (w/v) BSA
Add 0.88 g NaCl (MW 58.4) and 0.5% (w/v) BSA to 80 mL
0.01 M sodium phosphate, pH 7.4. Mix thoroughly and adjust
the volume to 100 mL with 0.01 M sodium phosphate pH 7.4.
Buffer E
PBS pH 7.4 with 0.1% (w/v) BSA
Add 0.88 g NaCl (MW 58.4) and 0.1% (w/v) BSA to 80 mL
of 0.01 M sodium phosphate pH 7.4. Mix thoroughly and
adjust the volume to 100 mL with 0.01 M sodium phosphate,
pH 7.4.

### Experimental Procedure for Functionalization

50 μl Dynabeads M-280 Tosylactivated (Invitrogen cat. 142.03) were placed in round-bottomed Eppendorf tubes and washed twice with 500 μl borate buffer (0.1 M borate buffer pH 9.5), for 5 minutes under gentle stirring on a wheel. The beads were collected on a magnetic device for 10 minutes, then the buffer was discarded and the beads were resuspended in 50 μl borate buffer. 30 μl of the bait phage from stock 1×10¹² TU/ml, i.e. 3×10⁶ phage clones, in borate buffer were added and then 60 μl borate buffer and 60 μl ammonium sulphate buffer (3M pH 7.4) were added. The tube was incubated at 37° C. for 24 h under gentle stirring in a rotating tumbler mixer with 30° inclination. The beads were separated on the magnetic device for 10 minutes, the supernatant was discarded and the beads were washed twice with 500 μl PBS buffer pH 7.4+1% BSA for 5 minutes under stirring on a wheel. Then 500 μl blocking buffer (PBS pH 7.4+4% BSA) were added and the beads were stirred on a wheel for 2 h at room temperature (RT). The beads were separated on the magnetic device for 10 minutes, the supernatant was discarded and the beads were washed twice with 500 μl PBS buffer pH 7.4+1% BSA for 5 minutes in a rotating tumbler mixer. Finally, the beads were resuspended in 200 μl PBS and left to stand at 4° C. (Dynabeads-bait phage). Three different stocks were prepared.

ELISA Test for Verifying the Functionalization of Dynabeads with the Bait Phage

In order to verify proper functionalization of the beads with the bait phage, an ELISA test was carried out by using an anti-phage M13 antibody. In brief, 20 μl functionalized beads were washed twice with 500 μl washing buffer (PBS pH 7,4+0.5% Tween 20) for 5 minutes on a wheel. The beads were resuspended in 100 μl PBS and 70 μl anti-M13 HPR antibody (1:2500 in PBS+0.1% BSA+0.5% Tween 20) were added, and then the beads were incubated in a rotating tumbler mixer at 37° C. for 1 h. Then 5 washings were carried out in 500 μl di PBS 0.5% Tween. In the last step, the beads were resuspended in 250 μl TMB and incubated for 20 minutes on a wheel at room temperature (RT) in the dark. After complete development, the reaction was quenched with 31 μl 6N H2SO4. The beads were placed on the magnet for 10 minutes and then 200 μl of the in-toto solution and of a 1:10 dilution were read at 450 nm.

### Results Obtained:

TABLE 2

| | Reading values at 450 nm | |
|---|---|---|
| | Undiluted solution (toto) | Diluted 1:10 |
| Dynabeads- | 2.415 | 1.407 |
| bait phage | | |

Capture Test for P. aeruginosa with Phage System Dynabeads Phage-pVIII Capture Tests

For capture and detections tests, an ON culture in 5 ml of LB of isolation P. aeruginosa ATCC 27853 on a cetrimide agar plate was used. The bacteria were collected by centrifugation at 8000×g for 10 minutes and resuspended in an isovolume of PBS. The bacteria were diluted in PBS to obtain a bacterial stock with OD600 of 0.29 (equal to 10⁸ cells/ml). 20 μl Dynabeads-bait phage were washed twice on a wheel for 5 minutes with 500 μl PBS, then they were placed on the magnet for 10 minutes, the supernatant was discarded and they were resuspended in 1 ml PBS and then 100 μl of the bacterial suspension 10⁸ cells/ml (final concentration 10⁷ cells) or 100 μl PBS (negative control) were added.

The sample was placed in an inclined wheel at 37° C. for 30 minutes.

The beads-bait phage with any captured bacteria were separated on the magnetic device for 10 minutes and then the supernatant was taken out and the beads-phage-bacteria complex was resuspended in 50 μl PBS and used for recognition with phage-pIII-CF594. The uncaptured bacteria present in the supernatant were titred by means of an on-plate dilution method and count of CFUs on Cetrimide agar to evaluate the bacterial capture efficiency on the Dynabeads functionalized with the bait phage. All tests were carried out in triplicate.

The efficiency capture of the cells of P. aeruginosa with the Dynabeads-bait phage in the carried out tests (calculated as the percentage of input number of bacteria-number of bacteria in the supernatant=number of captured bacteria) was on average 90%.

### Engineering of the Reporter Phage (Phage-pIII-CF594)

### Construction of the Reporter Phage of Gram-Negative Bacteria

The sequence coding for the peptide KLAKLAKKLAKLAK, capable of binding the outer membrane of Gram (-) bacteria was cloned in frame to a short linker GGGS upstream of the gene p3 of the phagemid pSEX81 (ProGen) in order to allow pentavalent display thereof on the tip of the detecting phage-pIII. An exponential phase culture of E. coli TG1 transformed with the aforesaid recombinant phagemid was then superinfected with the helper phage Hyperphage (ProGen) to allow production of the phage-pIII virions redirected to the recognition of Gram (-) bacteria. Following purification by precipitation in NaCl/PEG solution, the phages-pIII were resuspended in PBS pH 7.4 at a target concentration of about 10¹³ phages/ml. Said redirected phages-pIII were conjugated to the fluorophore CF594.

### Conjugation of the Reporter Phage to the Fluorophore CF594

The method of conjugating an imaging agent, being CF594, to the capsid of the detecting phage-pIII is described below. A stock solution of 10 mM CF594 succinimide ester in DMSO at room temperature is prepared.

10 μl of the stock solution are added to 1 ml of solution containing the reporter phage-pIII in 0.1 M bicarbonate buffer at pH 8.3, under constant stirring with a magnetic stirrer. The succinimidyl ester group of the dye reacts with the amine groups of the capsid proteins of the phage to form a stable amide bond. The mixture is kept under stirring at room temperature overnight.

The functionalized phage is separated from the unconjugated fraction of CF594 by dialysis (cut-off 14 kDa) in PBS buffer. 3 cycles of dialysis of 8 h each were carried out using 1 ml of solution of bioconjugate to be dialyzed and 500 ml PBS.

The quantification of phages was carried out by reading the UV-Vis absorbance spectrum of the phages, obtaining values for the wavelengths of 269 nm e 320 nm and then applying the following formula:

$$Phage concentration ( virions / mL ) = ( A 269 - A 320 ) * 6 * 10 \hat{} 16 / bp phage genome = 2.04 E + 13$$

In order to quantify the CF594 conjugated to the phage, the following formula has been applied:

$$conc . CF 594 ( microM ) = A 594 / epsilon 594 = 8.91$$

Given the concentration of CF594 and the concentration of phages (virions/ml), the ratio CF594 molecules/phage is obtained, corresponding to 264.

### Verification of the Redirection of the Reporter Phage-pIII-CF594 by Cytofluorimetry

The redirection of the phages-pIII-CF594 to P. aeruginosa (and other Gram-negative bacteria) was verified by performing cytofluorimetry assays using a BioRad S3e Cell Sorter: 10¹⁰ virions of the phage-pIII-CF594 were incubated for 20-30 minutes with 10⁹ cells of P. aeruginosa PA14 resuspended in 1 ml PBS pH 7.4. The non-redirected phage M13K07 (New England Biolabs) conjugated to CF594 was used as negative control for the cytofluorimetric analyses. The MFI (mean fluorescence intensity) detected for the phage-pIII-CF594 resulted increased by over 4 times with respect to the control M13K07—CF594, thus validating the fact that the detecting phage had been redirected to PA14. The cytofluorimetric analysis also made it possible to ascertain that one third of the population of P. aeruginosa had a fluorescence intensity increased by ˜ one order of magnitude (1 Log) following addition of the reporter phage-pIII-CF594. This is an index of the capability of the detecting phage to mediate the accumulation of several fluorophores near the Gram (-) bacterial cell.

Capture and Detection Tests with Double Phage System (Dynabeads Bait Phage-pVIII+Reporter Phage-pIII-CF594)
Labeling with Reporter Phage-pIII-CF594

To the complex beads-phage-bacteria resuspended in 50 μl PBS and to the negative control sample (without bacteria), 2 μl phage-pIII-CF594 (labeled stock 1:1, 2.04E+13 phages/ml, 0.91 μM CF594, 246 fluo/phage) were added and placed in a rotating mixer for 40 minutes at 37° C., in the last 5 minutes of reaction 1 μl DAPI (stock 10 mg/ml) or SYTO9 were added to label the captured bacterial cells. Each sample was placed on a magnet for 10 minutes, the supernatant was discarded, gentle washing was carried out in 100 μl PBS at RT for 5 minutes without stirring. The sample were then placed again on the magnet for 10 minutes, the supernatant was discarded, and gentle resuspension was carried out in 50 μl PBS. 10 μl of each sample were placed on a slide, and the samples were displayed with an Olympus fluorescence microscope (filter 540/605 lens 40x and 100x) and the images were captured by means of a CCD camera.

### Comments on the Results

The results obtained with the labeling of the captured bacteria with the reporter phage pIII CF 594 indicate a specific recognition of the phage pIII on P. aeruginosa complexed on the Dynabeads functionalized with the bait phage.

1. A method for detecting an analyte in a sample,
wherein said analyte is selected from:
a cellular system selected from: microbe, parasite, eukaryotic cell, bacterium, virus; or
a molecular marker selected from: toxin, protein or nucleic acids;
said method comprising the steps of:
a) contacting the sample with first phages M13 (bait phages) bound to an appropriate support and comprising at least one peptide or polypeptide or fusion protein specific for recognizing the analyte, said peptide or polypeptide or fusion protein being exposed on the protein pVIII, thus achieving formation of first complexes comprising first phages M13 bound to the analyte (“bait phage-analyte” complexes);
b) separating the first phages M13 from the sample part that did not bind to the first phages M13;
c) adding, to the first phages M13, second phages M13 (reporter phages) comprising at least one peptide or polypeptide or protein for specific recognition of the analyte fused on the protein pIII, and at least one marker conjugated to the capsid of said second phages M13, said marker being selected from a fluorophore, a chromophore, an electrochemically active species or an electrochemiluminescence-active species, thus achieving formation of second complexes comprising first phages M13 and second phages M13 bound to the analyte in a sandwiched manner;
d) carrying out a washing to remove the second phages M13 that did not bind the analyte,
e) determining the derived signal generated by the markers of the second phages M13 of said second complexes.

2. The method according to claim 1, wherein the analyte is a bacterium.

3. The method according to claim 2, wherein the analyte is a Gram-negative bacterium.

4. The method according to claim 3, wherein the analyte is the Gram-negative bacterium P. aeruginosa.

5. The method according to claim 4, wherein the at least one peptide or polypeptide or fusion protein of step a) has the sequence SEQ. ID NO: 1 (QRKLAAKLT).

6. The method according to claim 4, wherein the at least one peptide or polypeptide or phage recognition protein of step c) has the sequence SEQ. ID NO: 2 (KLAKLAKKLAKLAK).

7. The method according to claim 1, wherein the marker is a fluorophore.

8. The method according to claim 7, wherein the fluorophore is CF594 (Biotium).

9. The method according to claim 1, wherein the support comprises magnetic microspheres (MNP), said magnetic microspheres being functionalized with said first phages M13.

10. The method according to claim 9, wherein said step of separating the first phages M13 from the sample part that did not bind to the first phages M13 takes place by magnetic capture of the magnetic microspheres.

11. The method according to claim 1, wherein the support is an electrode, a polymer or a silicon-based surface.

12. The method according to claim 11, wherein said step of separating the first phages M13 from the sample part that did not bind to the first phages M13 takes place by washing.
